(19) United States
(12) Patent Application Publication
(10) Pub. No.: US 2004/0076581 A1
(43) Pub. Date: Apr. 22, 2004

(54) Use of neuropeptide y1 receptor binding compounds in the treatment and diagnosis of cancer

(76) Inventor: Jean-Claude Reubi, Wabern (DE)

Correspondence Address: Barbara E Johnson, 700 Koppers Building, 436 Seventh Avenue, Pittsburgh, PA, 15219-1818, US

(21) Appl. No.: 10/432,645

(22) PCT Filed: Nov. 21, 2001
(86) PCT No.: PCT/EP01/13621

(30) Foreign Application Priority Data
Nov. 24, 2000 (EP) 00204183.8

(51) Int. Cl. A61K 51/00

(52) U.S. Cl. 424/1.49

(57) ABSTRACT

The present invention relates to the use of compounds that bind the neuropeptide Y₁ (NPY₁) receptor for the preparation of a pharmaceutical composition for the diagnosis or treatment of tumors expressing NPY₁ receptors, in particular breast cancer, ovarian cancer and glioblastoma. The invention also relates to the pharmaceutical compositions that contain such compounds.

The present invention relates to the diagnosis and treatment of tumors expressing NPY₁ receptors, in particular breast cancer, ovarian cancer and glioblastoma.

NPY is a member of a family of 36 amino acid long peptides, including NPY, peptide YY (PYY) and pancreatic polypeptide (PP). Its main function is a neurotransmitter role and one of its best known actions in the CNS is stimulation of feeding behavior and inhibition of anxiety. Peripheral actions include effects on gastrointestinal motility and secretion, insulin release, renal secretion and vasoconstriction. The effect of NPY can be mediated by several NPY receptor subtypes, named Y₁-Y₆, from which Y₁, Y₂, Y₄ and Y₅ have been extensively characterized. Several NPY analogs, in particular Y₁ and Y₂ antagonists, are being developed for potential clinical use to treat feeding disturbances and anxiety.

Compared to other regulatory peptides, NPY has never been associated with human cancer. In the research that led to the present invention, the inventor has investigated whether there was a molecular basis for a putative NPY role in human tumors and/or for the development of NPY drugs for tumor targeting. NPY receptors were evaluated in one of the most frequent and harmful cancers, breast carcinoma. In more than 100 human breast tumor and metastasis samples, the expression of the two best investigated NPY receptor subtypes, Y₁ and Y₂, was studied using in vitro receptor autoradiographyl and in situ hybridization.

Based on the high density and high incidence of Y₁ the inventor contemplated that breast cancers represent an important target for NPY-related drugs. It was found that in analogy to other overexpressed peptide receptors, breast tumors and metastases can be targeted with radiolabeled Y₁ analogs for diagnostic in vivo scintigraphic tumor detection or for receptor-mediated radiotherapeutic treatment of these tumors in a similar manner as described for somatostatin (Colmers & Bleakman, Trends Neurosci. 17, 373-379 (1994); Wettstein et al., Pharmacol. Ther. 65, 397-414 (1995)). Moreover, long-term treatment with Y₁-selective analogs (Hökfelt et al., Neuropharmacology 39, 1337-1356 (2000); Walsh, J. H. Gastrin. in Gut Peptides: Biochemistry and Physiology (eds. Walsh, J. H. & Dockray, G. J.) 75-121 (Raven Press, New York, 1994)) is of considerable interest when NPY affects the proliferation of tumors.

It was found according to the invention that the neuropeptide Y₁ receptor is exclusively expressed on tumor tissue either in combination with the Y₂ receptor or alone, whereas healthy tissue only expresses the Y₂ receptor.

The invention according to a first aspect thereof thus relates to the use of compounds that bind the neuropeptide Y₁ (NPY₁) receptor for the preparation of a diagnostic or therapeutic composition for the diagnosis or treatment of tumors expressing NPY₁ receptors, in particular breast cancer, ovarian cancer and glial tumors.

Targeting these tumors is a powerful tool, not only in diagnosing such tumors but also in supporting an effective therapy. As a matter of fact, in order to be able to achieve a specific therapy for the control of such tumors, the detection and localization of these tumors, and in particular of the metastases thereof, in an early stage of their development is of utmost importance. Various requirements have to be imposed on an agent that is used in such a diagnostic method, such as non-toxic, no adverse influence on the host resistance and/or on the therapeutic treatment, well detectable and highly selective. The required high selectivity means that the diagnostic agent, after having been introduced into the body, must accumulate more strongly in the target tumors to be detected or visualized than in surrounding tissues. This selectivity, i.e. a comparatively stronger concentration of the diagnostic agent in the target tumors compared with non-target tissues, enables the user to correctly diagnose the malignancy. In order to be detectable from outside the body, the diagnostic agent should be labeled, preferably with a radionuclide or with a paramagnetic metal atom. In the former case, the radioactive radiation can be detected by using a suitable detector (scanning). Modern techniques in this field use emission tomography; when gamma radiating isotopes are used, the so-called single. photon emission computerized tomography (SPECT) may be applied. The use of paramagnetic diagnostic agents enables a detection by means of imaging by magnetic resonance.

For diagnosis, the compound binding the NPY₁ receptor is thus labeled with one of the following markers:

(a) a radioactive metal isotope selected from the group consisting of ^{99m}Tc, ²⁰³Pb, ⁶⁷Ga, ⁶⁸Ga, ⁷²As, ¹¹¹In, ^{113m}In, ⁹⁷Ru, ⁶²Cu, ⁶⁴Cu, ⁵²Fe, ^{52m}Mn, ¹⁷⁷Lu and ⁵¹Cr, or

(b) with a paramagnetic metal atom selected from the group consisting of Cr, Mn, Fe, Co, Ni, Cu, Pr, Nd, Sm, Yb, Gd, Tb, Dy, Ho and Er, or

(c) with a radioactive halogen isotope, selected from ¹²³I, ¹³¹I, ⁷⁵Br, ⁷⁶Br, ⁷⁷Br and ⁸²Br.

The thus labeled compound is administered to the subject to be diagnosed in an amount sufficient to be visualized by external imaging, by radioactive scanning or by magnetic resonance imaging, to determine the targeted sites in the body of said being in relation to the background activity, in order to allow detection and localization of the tumors in the body Such amount will usually vary between 1 and 20 μg.

For treatment, the compound that binds the NPY₁ receptor per se may have a therapeutic effect, or may be coupled to another molecule that has a therapeutic effect or to a radioisotope, selected from the group consisting of ^{114m}In, ¹⁸⁶Re, ¹⁸⁸Re, ⁷⁷As, ⁹⁰Y, ⁶⁶Ga, ⁶⁷Cu, ¹⁶⁹Er, ^{117m}Sn, ¹²¹Sn, ¹²⁷Te, ¹⁴²Pr, ¹⁴³Pr, ¹⁹⁸Au, ¹⁹⁹Au, ¹⁴⁹Tb, ¹⁶¹Tb ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, ¹¹¹Ag, ¹²⁴I and ¹³¹I. The compound per se or the compound thus labeled is administered to the subject to be treated in an amount sufficient to be effective. Such amount lies usually between 20 and 1000 μg.

Examples of other therapeutic molecules are cytotoxic agents such as doxorubicin or 2-pyrrolino-doxorubicin, covalently linked to a Y₁ analog such as [Leu³¹, Pro³⁴]-NPY or a constrained form of it.

The Y₁ receptor binding compound itself is selected from the group consisting of the following compounds [Leu³¹, Pro³⁴]-NPY, [Leu³¹, Pro³⁴]-PYY, Pro³⁴-NPY, Pro³⁴-PYY, NPY, PYY, Des Asn²⁹[Trp^{28,32}, Nva³⁴]-NPY(27-36) (Balasubramaniam, Peptides 18(3), 445-457 (1997)), [Pro³⁰, Tyr³², Leu³⁴]-NPY(28-36) (Leban et al., J. Med. Chem. 38, 1150-1157 (1995)), the dimer Bis (31/31′){[Cys³¹, Trp³², Nva³⁴]-NPY(31-36)) (Balasubramaniam, supra), SR120819A (Serradeil et al., FEBS lett. 225, 209-214 (1987)), BIBP3236 (Rudolf et al., Eur. J. Pharmacol. 271, R11-R13 (1994)), three compounds as described in Daniels et al., Proc. Natl. Acad. Sci. USA 92, 9067-9071 (1995): 383U91 of the formula

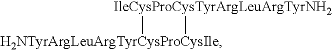

1120W91 of the formula

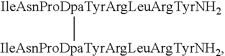

1229U91 of the formula

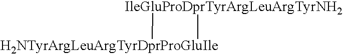

and arginine mimics.

Furthermore, the present invention relates to pharmaceutical compositions for the diagnosis or treatment of breast cancer, in particular in humans, comprising one or more compounds that bind the neuropeptide Y₁ receptor and a suitable carrier, diluent or excipient. The compound may be coupled to another therapeutically active molecule or label as described above.

It is frequently impossible to put a ready-for-use pharmaceutical composition at the disposal of the user, especially in the case of radiolabeled compounds due the often poor shelf life thereof and/or the short half-life of the radionuclide used. In such cases the user will carry out the labeling reaction with the radionuclide in the clinical hospital or laboratory. For this purpose the various reaction ingredients are then offered to the user in the form of a so-called “kit”. It will be obvious that the manipulations necessary to perform the desired reaction should be as simple as possible to enable the user to prepare from the kit the radioactive labeled composition by using the facilities that are at his disposal. Therefore the invention also relates to a kit for preparing a radiopharmaceutical composition.

Such a kit according to the present invention for preparing a radiopharmaceutical composition comprises (i) a NPY₁ receptor binding compound as defined above, to which compound, if desired, an inert pharmaceutically acceptable carrier and/or formulating agents and/or adjuvants is/are added, (ii) a solution of a salt or chelate of a metal isotope selected from the group consisting of ²⁰³Pb, ⁶⁷Ga, ⁶⁸Ga, ⁷²As, ¹¹¹In, ^{113m}In, ⁹⁷Ru, ⁶²Cu, ^{99m}Tc, ¹⁸⁶Re, ¹⁸⁸Re, ⁶⁶Cu, ⁵²Fe, ^{52m}Mn, ⁵¹Cr, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁶⁶Ho, ¹⁷²Tm, ¹⁶⁹Yb, ¹⁷⁵Yb, ¹⁷⁷Lu, ¹⁰⁵Rh and ¹¹¹Ag, and (iii) instructions for use with a prescription for reacting the ingredients present in the kit.

Preferably, when the compound is a peptide, the peptide compound to be used as an ingredient of the above kit has been derivatized by a reaction with a chelating agent.

Suitable chelating groups for chelating metal atoms are NₜS₍₄₋ₜ₎ tetradentate chelating agents, wherein t=2-4, or groups derived from ethylene diamine tetra-acetic acid (EDTA), diethylene triamine penta-acetic acid (DTPA), cyclohexyl 1,2-diamine tetra-acetic acid (CDTA), ethyleneglycol-0,0′-bis(2-aminoethyl)-N,N,N′,N′-tetra-acetic acid (EGTA), N,N-bis(hydroxybenzyl)-ethylenediamine-N,N′-diacetic acid (HBED), triethylene tetramine hexa-acetic acid (TTHA), 1,4,7,10-tetraazacyclododecane-N,N′,N″,N′″-tetria-acetic acid (DOTA), hydcroxyethyl-diamine triacetic acid (HEDTA), 1,4,8,11-tetra-azacyclo-tetradecane-N,N′,N″,N′″-tetra-acetic acid (TETA), substituted DTPA, substituted EDTA, or from a compound of the general formula

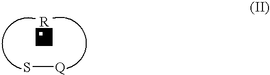

wherein R is a branched or non-branched, optionally substituted hydrocarbyl radical; which may be interrupted by one or more hetero-atoms selected from N, O and S and/or by one or more NH groups, and

Q is a group which is capable of reacting with an amino group of the peptide and which is preferably selected from the group consisting of carbonyl, carbimidoyl, N—(C₁-C₆)alkylcarbimidoyl, N-hydroxycarbimidoyl and N—(C₁-C₆)alkoxycarbimidoyl.

NₜS₍₄₋ₜ₎ chelating agents, wherein t=2-4, are preferably selected from

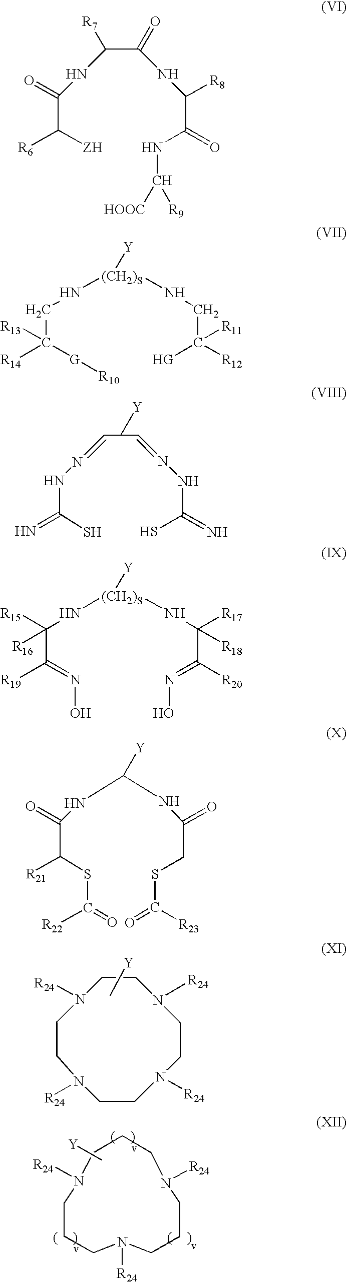

wherein:

R₆-R₂₀ are each individually hydrogen atoms or (C₁-C₄)alkyl groups, with the proviso that at least one of C₆ to C₉ is the symnbol Y;

R₂₁ is a hydrogen atom or a CO₂(C₁-C₄)alkyl group;

R₂₂ and R₂₃ are each individually (C₁-C₄)alkyl groups or phenyl groups;

v is 0 or 1;

s is 2 or 3;

R₂₄ is CH₂COOH or a functional derivative thereof;

A is (C₁-C₄)alkylene, if desired substituted with CO₂alkyl, CH₂COalkyl, CONH₂, CONHCH₂CO₂alkyl; phenylene,phenylene substituted by CO₂alkyl, wherein the alkyl groups have 1 to 4 carbon atoms;

G is NH or S;

Y is a functional group capable of binding with a free amino group of the peptide or with the spacing group;

and Z is S or O.

Said functional group Y preferably comprises isocyanato, isothiocyanato, formyl, o-halonitrophenyl, diazonium, epoxy, trichloro-s-triazinyl, ethyleneimino, chlorosulfonyl, alkoxycarb-imidoyl, (substituted or unsubstituted) alkylcarbonyloxycarbonyl, alkylcarbonylimidazolyl, succinimido-oxycarbonyl; said group being attached to a (C₁-C₁₀)hydrocarbon biradical.

Suitable examples of hydrocarbon biradicals are biradicals derived from benzene, (C₁-C₆)alkanes, (C₂-C₆)alkenes and (C₁-C₄)-alkylbenzenes.

Examples of suitable chelators of the general formula II are described in the international patent application WO 89/07456, such as unsubstituted or substituted 2-imino-thiolanes and 2-imino-thiacyclohexanes, in particular 2-imino-4-mercaptomethylthiolane.

Suitable examples of spacing groups, if present in the metal-labeled peptide molecule, are groups of the general formula

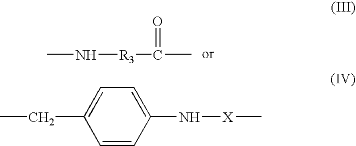

wherein R₃ is a C₁-C₁₀ alkylene group, a C₁-C₁₀ alkylidene group or a C₂-C₁₀ alkenylene group, and X is a thiocarbonyl group or a group of the general formula

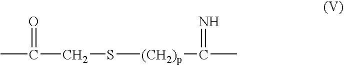

wherein p is 1-5.

Alternatively, peptide conjugates with avidin or biotin are formed, for example as described by Paganelli et al. (Int. J. Cancer 1988, 2, 121), Kalofonos et al. (J. Nucl. Med. 1990, 31, 1791) and Anderson et al. (FEBS LETT. 1991, 282/1, 35-40).

The resulting peptide conjugates provide a facility for firmly attaching the radionuclide in a simple manner. Suitable chelating agents for modifying peptides are described in detail hereinbefore. N-containing di- or polyacetic acids or their derivatives, such as the compounds mentioned before, have proved to be pre-eminently suitable for attaching various metal radionuclides, such as ¹¹¹In and ^{113m}In, to peptide molecules. The kit to be supplied to the user may also comprise the ingredient(s) defined sub (i) above, together with instructions for use, whereas the solution of a salt or chelate of the radionuclide, defined sub (ii) above, which solution has a limited shelf life, may be put to the disposal of the user separately.

In case the kit serves to prepare a radiopharmaceutical composition labeled with ^{99m}Tc, ¹⁸⁶Re or ¹⁸⁸Re, such a kit according to the present invention may comprise, in addition to the ingredient(s) defined sub (i) above, (ii) a reducing agent and, if desired, a chelator, and (iii) instructions for use with a prescription for reacting the ingredients of the kit with ^{99m}Tc in the form of a pertechnetate solution, or with ¹⁸⁶Re or ¹⁸⁸Re in the form of a perrhenate solution. If desired, the ingredients of the kit may be combined, provided they are compatible. The kit should comprise a reducing agent to reduce the pertechnetate or perrhenate, for example, a dithionite, a metallic reducing agent or a complex-stabilizing reducing agent, e.g. SnCl₂, Sn(II)-tartrate, Sn(II)-phosphonate or -pyrophosphate, or Sn(II)-glucoheptonate. The pertechnetate or perrhenate solution can simply be obtained by the user from a suitable generator.

When the radionuclide is present in the kit itself, the complex forming reaction with the derivatized peptide can simply be produced by combining the components in a neutral medium and causing them to react. For that purpose the radionuclide may be presented to the derivatized peptide in the form of a chelate bound to a comparatively weak chelator, as described hereinbefore.

When the kit comprises a derivatized peptide as defined hereinbefore and is intended for the preparation of a radiopharmaceutical composition, labeled with ^{99m}Tc, ¹⁸⁶Re or ¹⁸⁸Re, the radionuclide will preferably be added separately in the form of a pertechnetate or perrhenate solution. In that case the kit will comprise a suitable reducing agent and, if desired, a chelator, the former to reduce the pertechnetate or the perrhenate. As a reducing agent may be used, for example, a dithionite or a metallic reducing agent. The ingredients may optionally be combined, provided they are compatible.

Such a monocomponent kit, in which the combined ingredients are preferably lyophilized, is excellently suitable for being-reacted, by the user, with the radionuclide solution. As a reducing agent for the above-mentioned kits is preferably used a metallic reducing agent, for example, Sn(II), Ce(III), Fe(II), Cu(I), Ti(III) or Sb(III); Sn(II) is excellently suitable.

The peptide constituent of the above-mentioned kits, i.e. preferably the derivatized peptide, may be supplied as a solution, for example, in the form of a physiological saline solution, or in some buffer solution, but is preferably present in a dry condition, for example, in the lyophilized condition. When used as a component for an injection liquid it should be sterile, in which, when the constituent is in the dry state, the user should preferably use a sterile physiological saline solution as a solvent. If desired, the above-mentioned constituent may be stabilized in the conventional manner with suitable stabilizers, for example, ascorbic acid, gentisic acid or salts of these acids, or it may comprise other auxiliary agents, for example, fillers, such as glucose, lactose, mannitol, and the like.

The amount of the NPY₁ receptor binding compound in a pharmaceutical composition for diagnosis can be 1 to 20 μg. The composition can be administered through parenteral routes. The amount of the compound to be administered to the patient to be diagnosed is 1 to 20 μg.

The amount of the NPY₁ receptor binding compound in a therapeutic composition can be 20 to 1000 μg. The composition can be administered through parenteral routes. The amount of the compound to be administered to the patient to be treated is 20 to 1000 μg.

The invention furthermore relates to a method for diagnosing breast cancer in humans, comprising administration of a diagnostic pharmaceutical composition of the invention to a patient and visualizing the presence of the marker in the breast tumor, ovarian cancer or glial tumors.

It is another objective of the present invention to-provide a method of intraoperatively detecting and localizing tumors expressing NPY₁ receptors, in particular in breast, ovarian and glial tissues, which in healthy condition do not express NPY₁-receptors, in the body of a human being.

This objective can be achieved, according to a different aspect of the present invention by (i) administering to said being a composition comprising, in a quantity sufficient for detection by a gamma detecting probe, an active substance, consisting of a compound binding the neuropeptide Y₁ receptor labeled with ¹⁶¹Tb, ¹²³I or ¹²⁵I, and thereupon (ii), after allowing the active substance to be bound and taken up in said tumors and after blood clearance of radioactivity, subjecting said being to a radioimmunodetection technique in the relevant area of the body of said being, by using a gamma detecting probe.

Also subject of the invention is a method for treating breast cancer in humans, comprising injection of a therapeutic composition as described above to a patient in an amount sufficient to be effective in combating or controlling the tumor.

The present invention will be further elucidated in the example that follows and that is given for illustration purposes only and does not constitute any limitation to the invention.

In the example reference is made to the following figures:

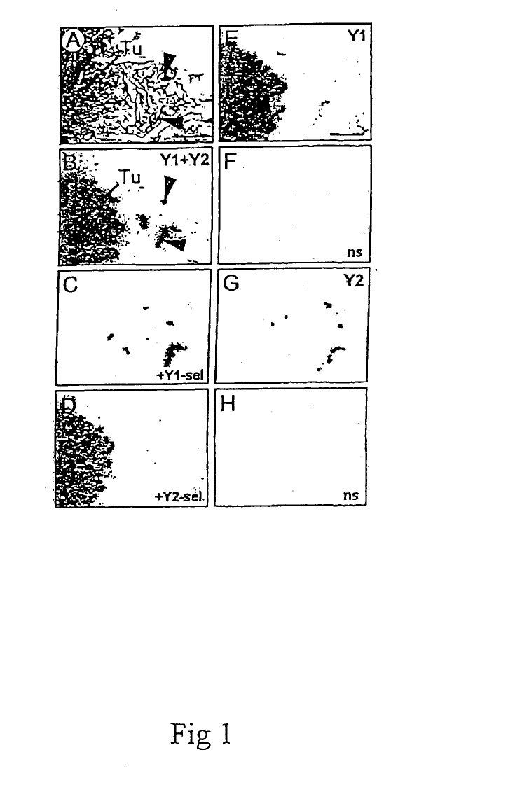

FIG. 1 NPY receptors in breast carcinoma and adjacent normal breast.

A: Hematoxylin-eosin staining showing tumor (Tu) and normal breast (arrowheads). Bar=1 mm.

B: Autoradiogram showing total binding of ¹²⁵I-PYY with strong labeling of tumor and breast.

C: Autoradiogram showing ¹²⁵I-PYY binding in presence of 25 nM of the Y₁-selective [Leu³¹, Pro³⁴]-NPY Complete displacement of the radioligand is seen in tumor but not in breast.

D: Autoradiogram showing ¹²⁵I-PYY binding in presence of 25 nM of the Y₂-selective PYY(3-36). Complete displacement is seen in breast but not in tumor.

E: Autoradiogram showing total binding of the Y₁-selective radioligand ¹²⁵I-[Leu³¹, Pro³⁴]-PYY. The tumor is strongly labeled, the breast tissue is not or only very weakly visible.

F: Autoradiogram showing non-specific binding ¹²⁵I-[Leu³¹, Pro³⁴]-PYY (in presence of 25 nM [Leu³¹, Pro³⁴]-NPY).

G: Autoradiogram showing total binding of the Y₂-selective radioligand ¹²⁵I-PYY(3-36). Tumor is not seen but adjacent breast tissue is labeled.

H: Autoradiogram showing non-specific binding of ¹²⁵I-PYY(3-36) (in presence of 25 nM of PYY(3-36)).

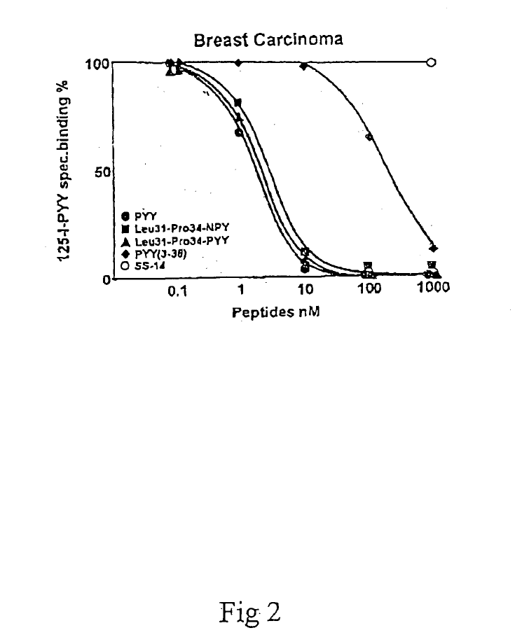

FIG. 2 Competition curves showing Y₁ in human breast carcinoma. The graph shows high affinity displacement of ¹²⁵I-PYY by PYY, [Leu³¹, Pro³⁴]-NPY, and [Leu³¹, Pro³⁴]-PYY, but not by PYY (3-36). Somatostatin (SS-14) is inactive.

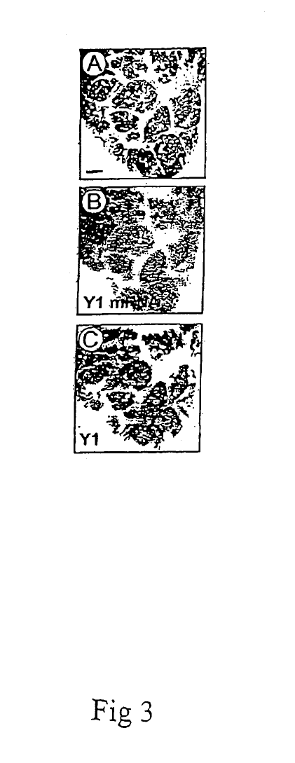

FIG. 3 Y₁ mRNA detected by in situ hybridization in an Y₁-expressing breast tumor.

A: Hematoxylin-eosin stained section showing breast carcinoma. Bar=1 mm.

B: Autoradiogram showing Y₁ mRNA in the tumor tissue. Non-specific labeling (in presence of a 20 fold excess of the corresponding probe) is negligible.

C: Autoradiogram showing binding of I-[Leu³¹, Pro³⁴]-PYY in the same tumor tissue.

### EXAMPLE

### Introduction

In this example it is investigated whether receptors for neuropeptide Y (NPY), a neurotransmitter with yet no established link to human cancer, can be overexpressed in human tumors. More than 100 samples of human breast carcinomas and adjacent breast tissues were tested for their expression of the NPY receptor subtypes Y₁ and Y₂ using in vitro receptor autoradiography with universal as well as subtype-selective analogs and in situ hybridization of Y₁ and Y₂ mRNA.

### Materials and Methods

### Patient Tissues

Breast tissue samples with primary breast neoplasias were obtained from 95 patients, aged 36 to 91, who were operated on in several institutions. Tissue samples were kept frozen at −80° C. The diagnosis was reviewed and formulated by use of cryostat sections, according to the WHO guidelines stated by Tavassoli (General considerations. in Pathology of the breast 1st edn (ed. Tavassoli, F. A.) 25-62 (Appleton & Lange, Norwalk, 1992)). In the main group of 89 patients, 61 (69%) showed an invasive ductal carcinoma.

In an additional group of six patients (4 ductal and 2 lobular breast carcinomas), tissue samples obtained from the primary tumor and from all the axillary [ metastases were investigated.

### NPY Receptor Autoradiography

Twenty-μm thick cryostat sections of the tissue samples were processed for NPY receptor autoradiography as described in detail previously for other peptide receptors (Reubi, J. C. et al. Cancer Res. 50, 5969-5977 (1990)). One radioligand used was ¹²⁵I-PYY (2′000 Ci/mmol; Anawa, Wangen, Switzerland), known to specifically label NPY receptors. For autoradiography, tissue sections were mounted on precleaned microscope slides and stored at −20° C. for at least 3 days to improve adhesion of the tissue to the slide. Sections were then processed according to Dumont et al. (J. Neurosci. 13, 73-86 (1993)). They were first preincubated in 119 mM NaCl, 3.2 mM KCl, 1.19 mM KH₂PO₄, 1.19 mm MgSO₄, 25 mM NaHCO₃, 2.53 mM CaCl₂×2H₂O and 10 mM D-glucose, pH 7.4 (preincubation solution), for 60 min at room temperature. The slides were then incubated in a solution containing the same medium as the preincubation solution in which the following compounds were added: 0.1% bovine serum albumin, 0.05% bacitracin, pH 7.4, and the radioligand, at approximate concentration of 22 pM ¹²⁵I-PYY. The slides were incubated at room temperature with the radioligand for 120 min.

To estimate nonspecific binding, paired serial sections were incubated as described above, except that 25 nM PYY was added to the medium. In order to distinguish Y₁ from Y₂ subtypes, increasing amounts of nonradioactive NPY, [Leu³¹, Pro³⁴]-NPY, a Y₁-selective ligand, and PYY(3-36), a Y₂-selective ligand, were added to the incubation medium to generate competitive inhibition curves using successive sections. Additional analogs used in competition experiments included pancreatic polypeptide and PYY(13-36). In selected cases, displacements with a single concentration (25 nM) of each of the above mentioned peptides were performed.

On completion of the incubation, the slides were washed four times for 5 min each in ice-cold preincubation solution, pH 7.4. They were rinsed twice in ice-cold distilled water, and then dried under a stream of cold air at 4° C., apposed to ³H-Hyperfilms and exposed for 7 days in x-ray cassettes.

In order to further distinguish Y₁ from Y₂ receptors, all cases demonstrating binding with ¹²⁵I-PYY were evaluated with the Y₁-selective radioligand ¹²⁵I-[Leu³¹, Pro³⁴]-PYY and with the Y₂-selective 125I-PYY(3-36) (Gehlert et al., Neurochem. Int 21, 45-67 (1992); Gehlert & Gackenheiner Neurosci. 76, 215-224 (1997)). Identical experimental conditions as mentioned for ¹²⁵I-PYY were used.

The autoradiograms were quantified using a computer-assisted image processing system, as described previously (Reubi et al. (1990), supra; Markwalder & Reubi Can. Res. 59,1152-1159 (1999)). Tissue standards for iodinated compounds (Amersham) were used for this purpose. A tissue was defined as receptor-positive when the optical density measured in the total binding section was at least twice that of the nonspecific binding section (in the presence of 10⁻⁷ mol/L PYY).

In each experiments, Y₁ expressing tissue (rat cortex) and Y₂ expressing tissues (stratum oriens and radiatum of the rat hippocampus) (Dumont et al. (1993), supra) were included as positive controls.

### In Situ Hybridization Histochemistry

Y₁ and Y₂ receptor mRNA were identified in selected normal and tumoral breast tissue samples with in situ hybridization histochemistry on cryostat sections, as described in detail previously (Reubi et al, Cancer Res 54, 3455-3459 (1994)) Oligonucleotide probes complementary to nucleotides 493-529 or 850-879 (Malmström, R. E. et al., Regul. Pept. 75-76, 55-70 (1998)) of the human Y₁ receptor gene and to nucleotides 223-252 (Malmström et al. (1998), supra) of the human Y₂ receptor gene or 1008-1052 (Schwarzer et al., Mol. Pharmacol. 55, 6-13 (1998)) of the rat Y₂ receptor gene (that sequence having 96% homology to the corresponding human one) were synthesized and purified on a 20% polyacrylamide-8M urea sequencing gel (Microsynth, Balgach, Switzerland). They were labeled at the 3′-end by using [α³²P] dATP (>3,000 Ci/mmol [>111 TBq/mmol]; NEN, Life Science Products, Boston, Mass.) and terminal deoxynucleotidyltransferase (Boehringer, Mannheim, Germany) to specific activities of 0.9-2.0×10⁻⁴ Ci/mmol [33.3-74 GBq/mmol]. Control experiments were carried out as reported previously (Reubi et al. (1994), supra) with the probes used in the present study to determine the specificity of the hybridization signal obtained

### Results

Table 1 summarizes the NPY receptor incidence in breast tissue in the main group of 89 patients. NPY receptors were expressed in a total of 76 of the 89 breast carcinomas tested. NPY receptors were found in 58/66 tested ductal carcinomas (55/61 invasive, 3/5 in situ) and 13/15 of the tested lobular carcinomas. Of the special types of invasive carcinomas, 2/3 mucinous carcinomas, 2/2 medullary, 1/1 tubular and 0/2 apocrine carcinomas were NPY receptor-positive.

For Y₁ and Y₂ subtype characterization, the two approaches used in the present study, namely the use of ¹²⁵I-PYY and its displacement by unlabeled Y₁ and Y₂ subtype-selective analogs (Dumont et al. (1993), supra) or the use of the two Y₁- and Y₂-selective radioligands (Gehlert & Gackenheimer (1997), supra) gave congruent results: Y₁ was the predominantly expressed receptor subtype in NPY receptor-positive tumors, with 100% incidence of this subtype in receptor-positive tumors, while Y₂ was only found in 24% of the cases (Table 1).

### Table 1

Incidence of NPY receptors Y₁ and Y₂ in human breast tissue

| | | | Cases with |
|---|---|---|---|
| | NPY-R | Differentiation by Y₁ | focal R |
| Tissue | Incidence* | and/or Y₂ expression** | distribution |
| breast | 76/89 | “Y₁-type of tumor***: 58/76 | 21/58 (36%) |
| carcinomas | (85%) | (76%) | Y₁: 3/18 |
| | | “mixed Y₁/Y₂ tumor type: 18/76 | (17%) |
| | | (24%) | Y₁: 10/18 |
| | | “Y₂-type” of tumor: 0/76 (0%) | (55%) |
| | | | — |
| non- | 45/45 | “Y₂-only” breast type: | |
| neoplastic | (100%) | 19/45 (42%) | |
| breast | | “switch Y₂/Y₁” breast type: | |
| (ducts + | | 26/45 (58%) | |
| lobules) | | “Y₁-only” breast type: 0/45 | |
| | | (0%) | |

*detected with “universal” ligand ¹²⁵I-PYY
**detected with Y₁-selective (¹²⁵I-[Leu₃₁, Pro₃₄]-PYY) or Y₂-selective (¹²⁵I-PYY(3-36)) radioligands
***“Y₁-type” of tumor was defined as those tumors expressing predominantly Y₁ but not Y₄. 46 cases had only Y₁; the remaining 12 cases had a density of Y₂ amounting less than 10% of the Y₁ density.

In 58/76 (76%) of the receptor-positive tumors, Y₁ was found to be the only (46 cases) or the predominant (12 cases containing more than 90% of Y₁ compared to Y₂) , receptor subtype expressed, while in 24% both Y₁ and Y₂ were highly expressed, and in none of the tumors, Y₂ was found alone.

It was noticed that Y₁ was more often homogeneously distributed within the tumor than it was the case for the Y₂ receptor, which was expressed focally, i.e. in certain restricted areas of the tumor only, in 55% of the cases.. Even in tumors expressing concomitantly Y₁ and Y₂, no tumor area was found that expressed Y₂ alone; Y₂ was only found in areas where Y₁ was expressed.

In an additional group of six patients not listed in Table 1, from whom primary breast tumors could be obtained together with all lymph node metastases, it was identified that the six primaries as well as all the metastases were expressing NPY receptors (Table 2); Y₁ was present in all cases, Y₂ in a few cases only, both in primaries as wells as in metastases (Table 2).

TABLE 2

| | Primary tumor | | Metatases | | |
|---|---|---|---|---|---|
| | NPY receptor subtype density (dpm/mg tissue) | | | | |
| | Y₁ | Y₁ | | Y₁ | Y₂ |
| Case 1 | 7200 | — | Meta 1 | 4511 | — |
| (ductal Ca) | | | Meta 2 | 2005 | — |
| | | | Meta 3 | 1420 | — |
| | | | Meta 4 | 3179 | — |
| | | | Meta 5 | 4241 | — |
| Case 2 | 1398 | — | Meta 1 | 5388 | — |
| (lobular Ca) | | | Meta 2 | 5481 | — |
| | | | Meta 3 | 5416 | — |
| Case 3 | 12262 | — | Meta 1 | 9430 | — |
| (ductal Ca) | | | Meta 2 | 10138 | — |
| | | | Meta 3 | 12298 | — |
| | | | Meta 4 | 11041 | — |
| | | | Meta 5 | 10471 | — |
| | | | Meta 6 | 11844 | — |
| | | | Meta 7 | 11015 | — |
| | | | Meta 8 | 12366 | — |
| | | | Meta 9 | 9553 | — |
| Case 4 | 12542 | 6535 | Meta 1 | 12205 | — |
| (ductal Ca | | | Meta 2 | 9970 | — |
| | | | Meta 3 | 12102 | 8220 |
| | | | Meta 4 | 12278 | — |
| | | | Meta 5 | 11060 | 6901 |
| | | | Meta 6 | 11062 | 6957 |
| Case 5 | 9787 | 2879 | Meta 1 | 7195 | 3440 |
| (lobular Ca) | | | Meta 2 | 8512 | 4105 |
| Case 6 | 9445 | — | Meta | 8627 | 4561 |
| (ductal Ca) | | | | | |

Table 1 further shows that NPY receptors can be detected in all tested normal breast tissues as well. In 42% of the cases, the Y₂ receptor is expressed alone, whereas in none of the tested breast tissues the Y₁ receptor is expressed alone. However, in the remaining breast tissues, Y₁ and Y₂ can be expressed concomitantly (58%).

FIG. 1 is a typical and representative example of the NPY receptor expression in a sample containing a breast carcinoma surrounded by normal breast tissue. The breast carcinoma expresses Y₁ receptors only as shown by the labeling of the tumor by ¹²⁵I-PYY and its displacement with [Leu³¹, Pro³⁴]-NPY but not by PYY(3-36). These results are further confirmed by the additional experiments using two other radioligands: the tumor is labeled by the Y₁-selective ¹²⁵I-(Leu³¹, Pro³⁴]-PYY but not by the Y₂-selective ¹²⁵I-PYY(3-36). Conversely, in the same tissue sections, the surrounding breast expresses predominantly Y₂ receptors, as shown by the opposite rank order of potency of NPY analogs, namely the high affinity of labeled and unlabeled PYY(3-36).but low affinity of [Leu³¹, Pro³⁴]-NPY and [Leu³¹, Pro³⁴]-PYY.

FIG. 2 shows representative displacement curves using the universal ¹²⁵I-PYY radioligand and increasing concentrations of Y₁ and Y₂-selective analogs. While, in a typical Y₁-expressing breast tumor, the Y₁-selective [Leu³¹, Pro³⁴]-NPY and [Leu³¹, Pro³⁴]-PYY completely displace the radiotracer with high affinity, the Y₂-selective PYY(3-36) was inactive.

In situ hybridization for Y₁ and Y₂ mRNA was performed in cases selected for their high expression of the respective receptor proteins. Y₁ mRNA was consistently shown in the 12 investigated Y₁-type of tumors. Furthermore, it was possible to detect Y₂ mRNA in isolated Y₂-expressing tubules of the normal breast. FIG. 3 illustrates Y₁ mRNA in a breast tumor.

### Discussion

This example is the first evidence that the neuropeptide NPY plays a potential role in cancer. It is remarkable that a great majority, i.e. 85% of human breast cancers, have an often high expression of NPY receptors. In all cases, the NPY receptor subtype Y₁ is expressed, whereas Y₂ is only expressed in 24% of the cases, and, when it is expressed, it never represents the predominant subtype of the tumor. In the 24% of the cases with a mixed expression of Y₁ and Y₂, a much more focal, topographically restricted distribution can be recognized for Y₂ than for Y₁, emphasizing once more the predominance of Y₁ in tumors. Both ductal and lobular breast cancers as well as all lymph node metastases can express NPY receptors.

Among the numerous cloned NPY receptors, the Y₁, Y₂, Y₄, and Y₅ represent at the moment the only fully defined subtypes (Michel et al. XVI. International union of pharmacology recommendations for the nomenclature of neuropeptide Y, peptide YY, and pancreatic polypeptide receptors. Pharmacol. Rev. 50, 143-150 (1998)). There are several arguments showing that the subtypes detected during the present study correspond to Y₁ and Y₂. Pharmacological evidence for Y₁ expression in tumors include a) specific binding of ¹²⁵I-PYY that is fully displaced in the high affinity range by the Y₁-selective [Leu³¹, Pro³⁴]-NPY, but not by PYY(3-36), PYY(13-36) or pancreatic polypeptide, compounds known to have high affinity for Y₂ or Y₆ (Michel et al. (1998), supra; Dumont et al. (1993), supra); b) selective binding of ¹²⁵I-[Leu³¹, Pro³⁴]-PYY in the same tissues (Gehlert & Gackenheimer (1997), supra); c) ionic, i.e. Ca⁺⁺-dependence typical for Y₁ (Wieland et al., Regul. Pept. 75-76, 263-269 (1998)); d) Y₁ mRNA detected by in situ hybridization in the tumor tissues. Furthermore, with those techniques we could confirm in humans the data from previous animal reports showing Y₁-expression in vessels (Bao et al., Proc. Natl. Acad. Sci. USA 94, 1261-1266 (1997): Hökfelt et al., Brain Res. Rev. 26, 154-166 (1998)).

1. Use of compounds that bind the neuropeptide Y₁ (NPY₁) receptor for the preparation of a pharmaceutical composition for the diagnosis or treatment of tumors expressing NPY₁ receptors, in particular breast cancer, ovarian cancer and glioblastoma.

2. Use as claimed in claim 1, wherein the compound is coupled to another molecule.

3. Use as claimed in claim 2 for the preparation of a pharmaceutical composition for diagnosis, wherein the other molecule is a radioactive metal isotope selected from the group consisting of ^{99m}Tc, ²³Pb, ⁶⁷Ga, ⁶⁸Ga, ⁷²As, ¹¹¹In, ^{113m}In, ¹²³I, ¹⁷⁷Lu, ⁹⁷Ru, ⁶²Cu, ⁶⁴Cu, ⁵²Fe, ^{52m}Mn and ⁵¹Cr.

4. Use as claimed in claim 2 for the preparation of a pharmaceutical composition for diagnosis, wherein the other molecule is a paramagnetic metal atom selected from the group consisting of Cr, Mn, Fe, Co, Ni, Cu, Pr, Nd, Sm, Yb, Gd, Tb, Dy, Ho and Er.

4. Use as claimed in claim 2 for the preparation of a pharmaceutical composition for diagnosis, wherein the other molecule is a radioactive halogen isotope, selected from ¹²³I, ¹³¹I, ⁷⁵Br, ⁷⁶Br, ⁷⁷Br and ⁸²Br.

5. Use as claimed in claim 2, wherein the other molecule is a therapeutical molecule for the treatment of cancer.

6. Use as claimed in claim 5, wherein the molecule for the treatment of cancer is a radioisotope selected from the group consisting of ^{114m}In, ¹⁸⁶Re, ¹⁸⁸Re, ⁷⁷As, ⁹⁰Y, ⁶⁶Ga, ⁶⁷Cu, ¹⁶⁹Er, ^{117m}Sn, ¹²¹Sn, ¹²⁷Te, ¹⁴²Pr, ¹⁴³Pr, ¹⁹⁸Au, ¹⁹⁹Au, ¹⁴⁹Tb, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb ¹⁷⁷Lu, ¹⁰⁵Rh, ¹¹¹Ag, ¹²⁴I and ¹³¹I.

7. Use as claimed in claims 1-6, wherein the NPY₁ receptor binding compound is selected from the group consisting of [Leu³¹, Pro³⁴]-NPY, [Leu³¹, Pro³⁴]-PYY, Pro³⁴-PYY, NPY, PYY, Des Asn²⁹[Trp^{28,32}, Nva³⁴]-NPY(27-36), [Pro³⁰, Tyr³², Leu³⁴]-NPY(28-36), the dimer Bis (31/31′){[Cys³¹, Trp³², Nva³⁴]-NPY(31-36)}, SR120819A, BIBP3236, the compound 383U91 of the formula 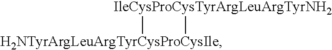
compound 1120W91 of the formula 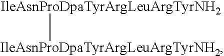
compound 1229U91 of the formula 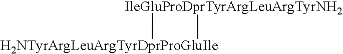
and arginine mimics.

8. Pharmaceutical composition for the diagnosis or treatment of NPY₁ receptor expressing tumors, in particular breast cancer, ovarian cancer or glioblastoma, in particular in humans, comprising one or more compounds that bind the neuropeptide Y₁ receptor and a suitable carrier, diluent or excipient.

9. Pharmaceutical composition as claimed in claim 8, wherein the compound is coupled to another molecule.

10. Pharmaceutical composition as claimed in claim 9 for the preparation of a pharmaceutical composition for diagnosis, wherein the other molecule is a radioactive metal isotope selected from the group consisting of ^{99m}Tc, ²⁰³Pb, ⁶⁷Ga, ⁶⁸Ga, ⁷²As, ¹¹¹In, ^{113m}In, ⁹⁷Ru, ⁶²Cu, ⁶⁴Cu, ⁵²Fe, ^{52m}Mn and ⁵¹Cr.

11. Pharmaceutical composition as claimed in claim 9 for the preparation of a pharmaceutical composition for diagnosis, wherein the other molecule is a paramagnetic metal atom selected from the group consisting of Cr, Mn, Fe, Co, Ti, Cu, Pr, Nd, Sm, Yb, Gd, Tb, Dy, Ho and Er.

12. Pharmaceutical composition as claimed in claim 9for the preparation of a pharmaceutical 5 composition for diagnosis,.wherein the other molecule is a radioactive halogen isotope, selected from ¹²³I, ¹³¹I, ⁷⁵Br, ⁷⁶Br, ⁷⁷Br and ⁸²Br.

13. Pharmaceutical composition as claimed in claim 9, wherein the other molecule is a therapeutical molecule for the treatment of cancer.

14. Pharmaceutical composition as claimed in claim 13, wherein the molecule for the treatment of cancer is a-radioisotope selected from the group consisting of ^{114m}In, ¹⁸⁶Re, ¹⁸⁸Re, ⁷⁷As, 90Y, ⁶⁶Ga, ⁶⁷Cu, ¹⁶⁹Er, ^{117m}Sn, ¹²¹Sn, ¹²⁷Te, ¹⁴²Pr, ¹⁴³Pr, ¹⁹⁸Au, ¹⁹⁹Au, ¹⁴⁹Tb, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, ¹¹¹Ag, ¹²⁴I and ¹³¹I.

15. Pharmaceutical composition as claimed in claims 8-14, wherein the NPY₁ receptor binding compound is selected from the group consisting of [Leu³¹, Pro³⁴]-NPY, [Leu³¹, Pro³⁴]-PYY, Pro³⁴-PYY, NPY, PYY, Des Asn²⁹[Trp^{28,32}, Nva³⁴]-NPY(27-36), [Pro³⁰, Tyr³², Leu³⁴]-NPY(28-36), the dimer Bis (31/31′){[Cys³¹, Trp³², Nva³⁴]-NPY(3l-36)}, SR120819A, BIBP3236, the compound 383U91 of the formula 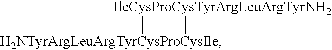
compound 1120W91 of the formula 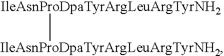
compound 1229U91 of the formula 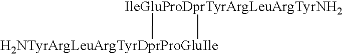
and arginine mimics.

16. Kit for preparing a radiopharmaceutical composition, comprising (i) a NPY₁ receptor binding compound, which, when the compound is a peptide, may be optionally derivatized, to which compound, if desired an inert pharmaceutically acceptable carrier and/or formulating agents and/or adjuvants is/are added, (ii) a solution of a salt or chelate of a metal selected from the group consisting of the radioactive isotopes ²⁰³Pb, ⁶⁶Ga, ⁶⁷Ga, ⁶⁸Ga, ⁷²As, ¹¹¹In, ^{113m}In, ^{114m}In, ⁹⁷Ru, ⁶²Cu, ^{99m}Tc, ¹⁸⁶Re, ¹⁸⁸Re, ⁶⁴Cu, ⁵²Fe, ^{52m}Mn, ⁵¹Cr, ⁷⁷As, ⁹⁰Y, ⁶⁷Cu, ¹⁶⁹Er, ^{117m}Sn, ¹²¹Sn, ¹²⁷Te, ¹⁴²Pr, ¹⁴³Pr, ¹⁹⁸Au, ¹⁹⁹Au, ¹⁴⁹Tb, ¹⁶¹Tb, ¹⁰⁹Pd. ¹⁶⁵Dy, ¹⁴⁹Pm, 151Pm, ¹⁵³Sm, ¹⁵⁷Gd, ¹⁶⁶Ho, ¹⁷²Tm, ¹⁶⁹Yb, 175Yb, ¹⁷⁷Lu, ¹⁰⁵Rh and ¹¹¹Ag, and (iii) instructions for use with a prescription for reacting the ingredients present in the kit.

17. A kit for preparing a radiopharmaceutical composition, comprising (i) a NPY₁ receptor binding compound, which, when the compound is a peptide, may be optionally derivatized, to which compound, if desired, an inert pharmaceutically acceptable carrier and/or formulating agents and/or adjuvants is/are added, (ii) a reducing agent, and, if desired, a chelator, said ingredients (i) and (ii) optionally being combined, and (iii) instructions for use with a prescription for reacting the ingredients of the kit with ^{99m}Tc in the form of a pertechnetate solution or with ¹⁸⁶Re or ¹⁸⁸Re in the form of a perrhenate solution.

18. A method of detecting and localizing NPY₁ receptor expressing tumors and their metastases in tissues, which in healthy condition do not contain NPY₁ receptors, in the body of a human being, which comprises (i) administering to said being a composition comprising, in a quantity sufficient for external imaging, a NPY₁ receptor binding compound, said compound being labeled with (a) a radioactive metal isotope selected from the group consisting of ^{99m}Tc, ²⁰³Pb, ⁶⁷Ga, ⁶⁸Ga, ⁷²As, ¹¹¹In, ^{113m}In, ⁹⁷Ru, ⁶²Cu, ⁶⁴Cu, ⁵²Fe, ^{52m}Mn and ⁵¹Cr, or (b) with a paramagnetic metal atom selected from the group consisting of Cr, Mn, Fe, Co, Ni, Cu, Pr, Nd, Sm, Yb, Gd, Tb, Dy, Ho and Er, or (c) with a radioactive halogen isotope, selected from ¹²³I, ¹³¹I, ⁷⁵Br, ⁷⁶Br, ⁷⁷Br and ⁸²Br, and thereupon (ii) subjecting said being to external imaging, by radioactive scanning or by magnetic resonance imaging, to determine the targeted sites in the body of said being in relation to the background activity, in order to allow detection and localization of said tumors in the body.

19. A method of intraoperatively detecting and localizing NPY₁ receptor expressing tumors in tissues, which in healthy condition do not contain NPY₁ receptors, in the body of a human being, which comprises (i) administering to said being a composition comprising, in a quantity sufficient for detection by a gamma detecting probe, an active substance, which consists of a compound that is labeled with ¹⁶¹Tb, ¹²³I or ¹²⁵I, and thereupon (ii), after allowing the active substance to be bound and taken up in said tumors and after blood clearance of radioactivity, subjecting said being to a radioimmunodetection technique in the relevant area of the body of said being, by using a gamma detecting probe.

20. A method for the therapeutic treatment of NPY₁ receptor expressing tumors in tissues, which in healthy condition do not contain NPY₁ receptors, in the body of a human being, which comprises administering to said being a composition comprising, in a quantity effective for combating or controlling tumors, a NPY₁ recptor binding compound labeled with an isotope selected from the group consisting of ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, ¹¹¹Ag, ¹²⁴I and ¹³¹I,

21. A method as claimed in any of the claims 18-20, characterized in that the tumors and the metastasis thereof to be detected, localized or therapeutically treated are selected from the group consisting of breast cancer, ovarian carcinoma and glioblastoma.
